(19) United States
(12) Patent Application Publication
(10) Pub. No.: US 2015/0273188 A1
(43) Pub. Date: Oct. 1, 2015

(54) SYSTEM AND METHOD FOR MANUFACTURING CATHETER BALLOONS

(71) Applicant: Resonetics, LLC, Nashua, NH (US)

(72) Inventors: Rong Gu, Hudson, NH (US); Timothy P. Malone, Freemont, CA (US); David Torres, Stoughton, MA (US)

(73) Assignee: RESONETICS, LLC, Nashua, NH (US)

(21) Appl. No.: 14/226,483

(22) Filed: Mar. 26, 2014

(51) Int. Cl. A61M 25/10

(52) U.S. Cl. CPC A61M 25/1029; A61M 25/10; A61M 2025/1031

(57) ABSTRACT

In some embodiments of the present disclosure, a method for manufacturing a polymeric medical balloon is provided which may include providing a mold, wherein the mold comprises a plurality of assembled parts configured for separation to remove a molded balloon, configuring the interior surface of a least a portion of at least one part of the mold with one or more micro-features, inserting a balloon preform within the mold, and expanding the preform within the mold to obtain an expanded balloon. The one or more micro-features of the mold produce one or more corresponding features on the surface of the expanded balloon, and the one or more corresponding features on the surface of the expanded balloon effect at least one of a depth and height between about 10 μm and about 500 μm such that the one or more corresponding features are configured as reference points for positional registration of the balloon for subsequent processing of the expanded balloon. The method may further include removing the expanded balloon from the mold.

### FIELD OF THE DISCLOSURE

Embodiments of the present disclosure are directed toward balloon catheters, and more specifically, the manufacture of such balloons, machining of such balloons using lasers (for example), and the placement of such balloon in a living body during a surgical procedure.

### BACKGROUND OF THE DISCLOSURE

Polymer catheter shafts and catheter balloons are used in an increasingly widening variety of medical procedures including vascular dilatation, stent delivery, drug delivery, sensors delivery and operation, as well as surgical cutting implements such as blades, and the like. The desired physical profiles of balloons used in these procedures vary according to the specific application.

For example, for drug delivery, sensor delivery, as well as similar applications, the surface of the balloon is often modified with micro-structures for certain desired functionality. In most cases, such micro-structures are added after the balloon is fabricated (e.g., by laser micromachining). The locations of those microstructures with respect to specific portions of the balloon are important due to such desired functionality (e.g., drug release dynamics), as well as for mechanical behavior of the balloon itself.

In balloon-expanded medical device applications (e.g., stents), it is important that the medical device be accurately positioned with respect to the body portion, of a balloon (or functionally similar devices). Failure to properly position it may result in a non-uniform expansion of or damage to the medical device and/or balloon.

In both examples, there is a need to have clear and well-defined reference positions on the surface of a balloon so that such reference positions can be used for further processing, including, for example, expandable, medical device placement, fabrication of microstructures, and the like.

### Summary of Some of the Embodiments

In some embodiments of the present disclosure, a method for manufacturing a polymeric medical balloon is provided which may comprise one or more of the following steps, and in some embodiments, a plurality of the steps, and in some embodiments, all of the steps. The method may include providing a mold, where the mold comprises a plurality of assembled parts configured for separation to remove a balloon after molding. The method may further include configuring the interior surface of a least a portion of at least one part of the mold with one or more micro-features, inserting a balloon preform within the mold, and expanding the preform within the mold to obtain an expanded balloon. The one or more micro-features of the mold may produce one or more corresponding features on the surface of the expanded balloon, and the one or more corresponding features on the surface of the expanded balloon effect at least one of a depth and height between about 10 μm and about 500 μm such that the one or more corresponding features are configured as reference points for positional registration of the balloon for subsequent processing of the expanded balloon. The method may further include removing the expanded balloon from the mold.

In some embodiments, a method for manufacturing a polymeric medical balloon is provided which may include one or more of the following steps, and in some embodiments, a plurality of the steps, and in some embodiments, all of the steps. Accordingly, the method may include providing a mold, where the mold comprises a plurality of assembled parts configured for separation to remove a molded balloon. A first part of the mold may be arranged immediately adjacent a second part of the mold forming a seam there between. The first part of the mold may be configured with a first radius at or adjacent the seam, and the second part of the mold may be configured with a second radius at or adjacent the same. In some embodiments, the first radius is either larger or smaller than the second radius to effect a radial mis-match on a molded balloon. The method may further include inserting a balloon preform within the mold, and expanding the preform within the mold to obtain an expanded balloon. The radial mis-match can be configured to establish a measurable difference of between about 10 μm and about 500 μm to effect a reference mark for positional registration of the balloon for subsequent processing of the expanded balloon. In some embodiments, the method may further include removing the expanded balloon from the mold.

In some embodiments, a polymeric balloon is provided which may include one or more micro-features arranged on the surface thereof. The one or more micro-features effect at least one of a depth and height between about 10 μm and about 500 μm, and may be configured as reference points for positional registration of the balloon for subsequent processing of the expanded balloon.

In some embodiments, a polymeric balloon having at least two sections is provided. The sections may include at least a first section and a second section, and a seam provided therebetween. The first section of the balloon may be configured with a first radius at or adjacent the seam, and the second section of the balloon may be configured with a second radius at or adjacent the seam. In some embodiments, the first radius is either larger or smaller than the second radius to effect a radial mis-match on a molded balloon, and the radial mis-match may establish a measurable difference of between about 10 μm and about 500 μm to effect a reference mark for positional registration of the balloon for subsequent processing of the expanded balloon.

One and/or another of embodiments herein disclosed may additional include one or more of the following additional features:
the one or more corresponding features on the surface of the expanded balloon effect at least one of a depth and height between about 50 μm and about 150 μm;
the one or more corresponding features comprise one or more of a groove, a recess, and/or a protrusion, such recesses or protrusions may include cylindrical, spherical or otherwise round shapes;
the one or more corresponding features comprise a plurality of radial and/or longitudinal grooves;
the one or more micro-features which produce the one or more grooves, recesses, and/or protrusions, are provided at or adjacent a seam between two parts of the mold, such recesses or protrusions may include cylindrical, spherical or otherwise round shapes;
the one or more micro-features which produce the one or more grooves, recesses, and/or protrusions, are provided at or adjacent a transitional area of the mold, such recesses or protrusions may include cylindrical, spherical or otherwise round shapes;
subsequent processing may include at least one of: modification of the balloon, and placement of the balloon in a surgical procedure, and, modification of the balloon may comprise laser micromachining of the balloon;
the one or more micro-features comprise a plurality of micro-features, and some of the plurality of micro-features effect second corresponding features in the surface of the molded balloon which comprise drug depots; and
the difference between the first radius and the second radius is between about 50 μm and about 150 μm;

These and other embodiments, features, objects and/or advantages of the present disclosure will become even more clear with reference to the following detailed description and attached drawings, a brief description of which is provided below.

### BRIEF DESCRIPTION OF THE DRAWINGS

One of skill in the art will appreciate that for the subject disclosure, modifications and/or feature shown for one and/or another of balloons (and/or molds) are exaggerated to provide clarity and visibility. Moreover, the subject disclosure figures are not drawn to scale.

FIG. 1 is an illustration of a prior art polymeric medical balloon.

FIGS. 2A-G are illustrations of prior art polymeric medical balloons of various shapes and sizes.

FIG. 2H shows placement of a stent on a balloon in the prior art, illustrating the difficulty in finding a well-defined reference feature for exact positioning of the stent in axial direction with respect to the balloon.

FIG. 2I shows the difficulty in locating a reference feature (here, a transition) for enabling easy targeting of an area for machining a balloon, according to the prior art.

FIG. 3A is a cross-section of a polymeric medical balloon according to some embodiments of the disclosure.

FIG. 3B is a cross-section of a polymeric medical balloon according to some embodiments of the present disclosure, where one part of the medical balloon includes a first diameter/radius, and a second part of the balloon includes a second diameter/radius which is either larger or smaller than the diameter/radius of the first part.

FIG. 3C is a cross-section of a polymeric medical balloon according to some embodiments of the present disclosure which provides a seam-line reference portion arranged between one part of the medical balloon a second part of the balloon.

FIG. 3D is a cross-section of a polymeric medical balloon according to some embodiments of the present disclosure which provides a seam-line reference portion arranged between one part of the medical balloon a second part of the balloon, where the seam-line reference portion comprises one or more (and in some embodiments a plurality) of longitudinal and/or circumferential grooves or ridges.

FIG. 3E is a cross-section of a polymeric medical balloon according to some embodiments of the present disclosure which provides a reference portion arranged at or adjacent a transitional portion of the balloon, where the reference portion comprises one or more (and in some embodiments a plurality) of longitudinal and/or circumferential grooves or ridges.

FIG. 4 illustrates a balloon mold, according to some embodiments, which may be made in at least two separable parts/pieces (for example) to allow removal of the expanded balloon, where the parts may be joined in either the sagittal plane or in the transverse plane.

FIG. 5 shows a balloon according to some embodiments, having a transition edge which can be used as a locating feature for enabling easy targeting of an area of the balloon for machining, according to some embodiments.

### DETAILED DESCRIPTION OF SOME OF THE EMBODIMENTS

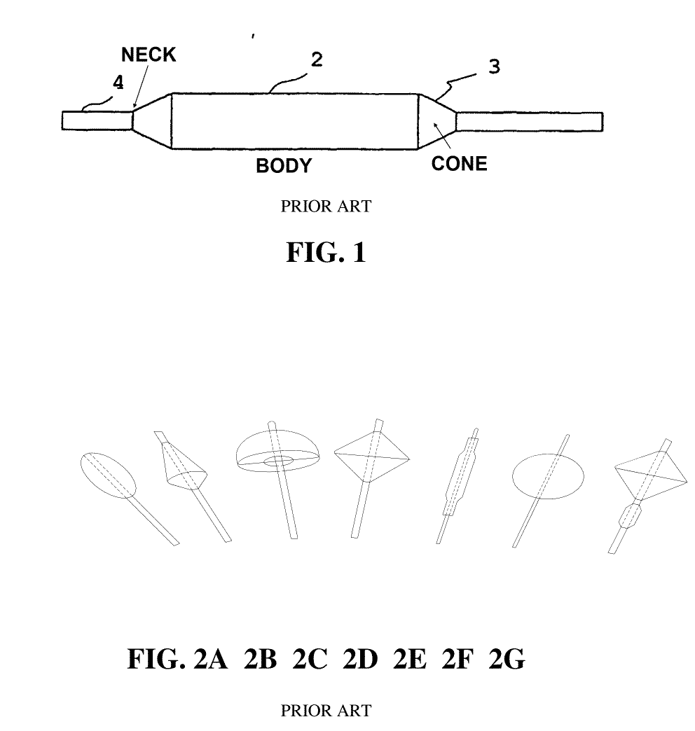

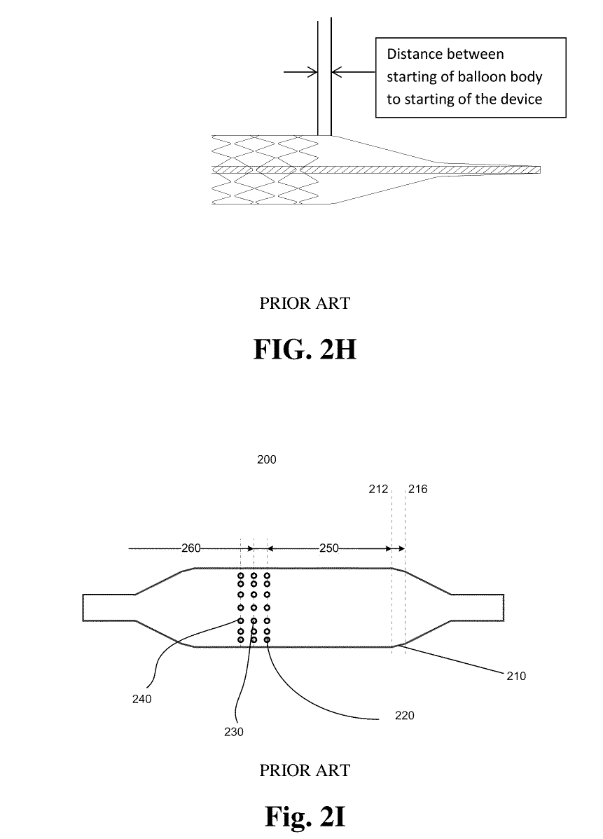

Commercial balloons are formed of a wide variety of materials, including polymeric materials, a general overview of such is provided in FIGS. 1 and 2A-G. As shown in FIG. 1, a tubular balloon (1) is illustrated having a body (2), first portion (4) connected to the body (2) via transitional portion (3), which may be a conical section. Such polymeric materials include PET, nylons, polyurethanes, and various block copolymer thermoplastic elastomers. A common method for forming balloons is to expand a heated, tubular balloon preform (parison) into an external mold by applying gas pressure. This forces the preform to conform to the inner surface of the mold to obtain the desired shape. To facilitate forming the balloon, the mold shape is optimized for the removal of material. Such optimization requires reducing obstacles, and designing a shape or form that does not inhibit material flow. FIG. 2H shows placement of a stent on a balloon in the prior art, illustrating the difficulty in finding a well-defined reference feature for exact positioning of the stent in axial direction with respect to the balloon. Techniques for manufacturing polymeric medical balloons comprise blow-molding, examples of which can be found in U.S. Pat. No. 6,465,067 (“the '067 patent”), herein incorporated by reference.

Balloons made according to the techniques outlined in the '067 patent may be laser machined to effect, for example, one or more orifices in the balloon. For example, as illustrated in FIG. 2I, a balloon 200 includes a transition portion between cylindrical and conical sections, which is sometimes used to help locate an area for further laser machining to be effected. Balloon 200 is placed approximately in the field of view of an operator or vision system. A transition portion/edge 210 between cylindrical and conical sections is attempted to be located. Laser processing is then performed at a certain distance offset from the edge definition 212, 216, to effect a first laser pattern 220 (e.g., a plurality of orifices circumferentially placed around the perimeter of the balloon). Other machining locations 230, 240 can be so located, e.g., a location offset by a predetermined distance 260 from the a first laser pattern or edge definition 212, 216.

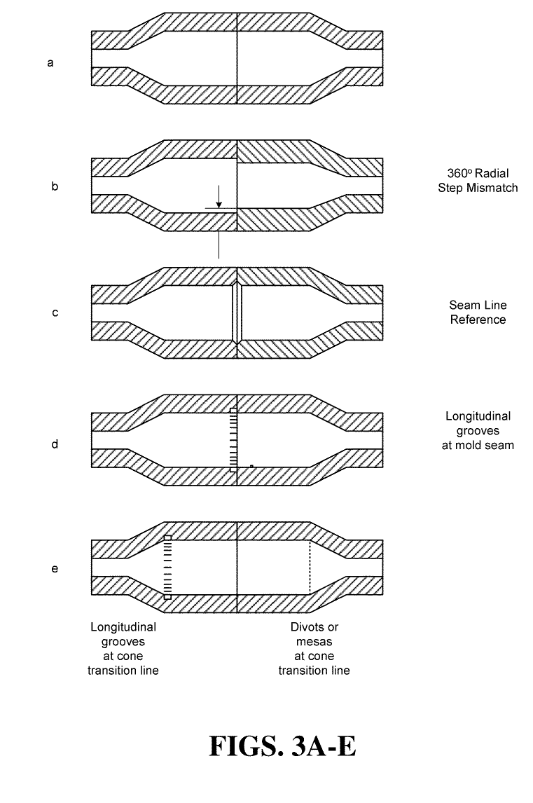

In some embodiments of the present disclosure, features may be created in the balloon during manufacture of the balloon which enable for easy location for effecting further processing/laser machining or placement of other structure/devices (e.g., a stent) relative to the balloon. For example, as shown in FIG. 3A, balloons may be between approximately 1 and 500 mm in length (and in some embodiments, between about 3 mm and about 300 mm in length), include an outside diameter of between about 1 and about 10 mm (and in some embodiments, between about 0.5 mm and about 100 mm). Of course, molds to manufacture such balloons include corresponding dimensions to produce such balloons. The walls of the balloons, according to some embodiments, are between about 25 and about 250 microns (in some embodiments, the wall thickness may be between about 10 and 500 microns, and in some embodiments, between about 50 and 100 microns).

To avoid tears and bursting of the balloon during the forming process, as well as to achieve predictable wall thickness, in some embodiments, the mold's inner surface is configured to be without surface imperfections or roughness and include transitioning areas for transitioning the mold from segment to another. While in current commercial balloons manufactured from polymeric materials, the smooth transition areas (e.g., (from body to cone, from cone to neck, etc., see FIG. 1) cannot provide precise references for subsequent process registration.

Accordingly, in some embodiments of the present disclosure, a balloon may be formed of any material which can be radially and longitudinally expanded out of a tubular parison. In such embodiments, the balloon mold may be made from metal (e.g., stainless steel, titanium, brass, and other alloys) or polymers (such as polycarbonate and polyolefins).

### EXAMPLE

### Cylindrical Balloon with Conical Ends

The following example is for illustrative purposes only and is not meant to be limiting. The concepts for the following process (and resulting products as well as molds and systems to produce the same) are for exemplary purposes only for applicability to, for example, other balloon shapes.

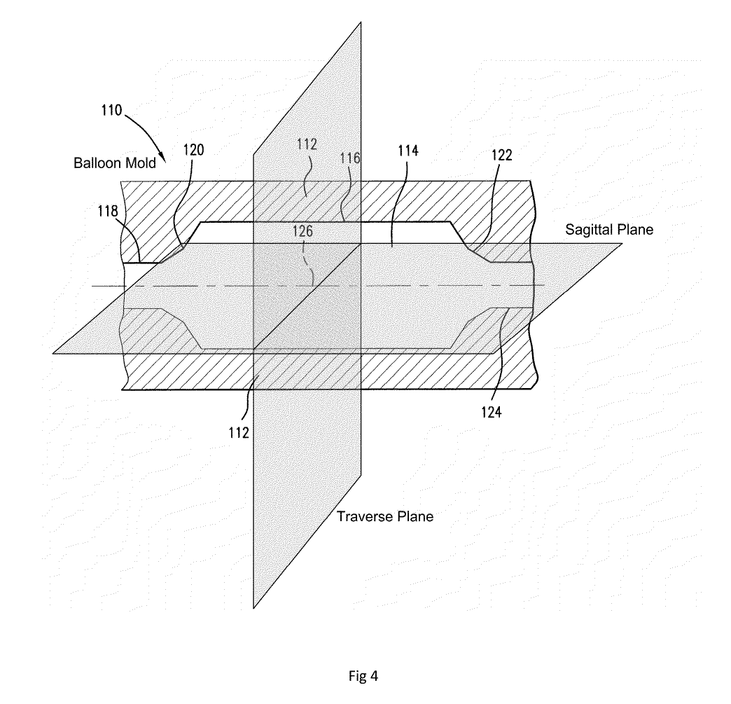

A mold cavity for a cylindrical balloon with conical ends includes a generally cylindrical inner molding surface in the middle, as well as distal and proximal cone shape surfaces in both ends. The mold is typically made in a plurality of parts/pieces, specifically, in some embodiments, at least two separable parts to allow for (for example), removal of the expanded balloon. The parts may be joined either in sagittal plane or in transverse plane (for example), as shown in FIG. 4 (for example).

In some embodiments, one or more intentional and specifically placed reference features may be provided on the outer surface of the balloon by, for example, replicating corresponding features on the inner surface of the mold.

FIG. 3B illustrates some embodiments according to the present disclosure, in which the two halves of the mold are fabricated to produce a differential in the internal diameter (ID) of the mold at the seam-line (for example). Such a differential may range between about 10 and about 200 microns, and in some embodiments, between about 50 and 125 microns, and in some embodiments, about 100 microns. In some embodiments, the ID differential is configured such that it does not affect balloon formation.

Moreover, in some embodiments, the height of the resulting reference line on the OD of the balloon is configured to be of a size which is relatively smaller (and in some embodiments, substantially smaller) than normal size variations in balloon forming; in other words, the presence of the reference line, in some embodiments, should not change the performance of the balloon with respect to any of its functionalities. For example, in some embodiments, two parts of a balloon mold are chamfered at their matching surfaces, so that a precise and defined reference line is formed on the OD of the mold. The differential, according to some embodiments, establishes a detectable, precise reference line on the outer diameter (OD) of the balloon. The width of this reference-line at the seam may be between about 10 microns and about 1 mm, and in some embodiments, between about 100 and 750 microns, and in some embodiments, about 500 microns. The reference-line, in some embodiments, is configured for alignment of the balloon for further processing (e.g., laser micromachining). FIG. 3C illustrates an example of these embodiments.

In some embodiments, modifications may be made to the mold surface as one or more, and in some embodiments, a series of longitudinal grooves or ridges, in contrast to other noted embodiments which utilize a continuous (or substantially continuous) difference in mode ID circumferentially along 360° (or a majority or substantially all of the circumference) of the mold ID. The groove(s)/ridge(s) may be arranged at balloon locations in need of a positional reference (for example). Such a mark(s) may be configured for referencing at least one of, and in some embodiments, both longitudinal and circumferential positions on the surface of a balloon. In such embodiments, the depth or height of such a groove(s) or ridge(s), respectively, can be between about 10 and 250 microns, and in some embodiments between about 50 and 150 microns, and in some embodiments, about 100 microns. In some embodiments, the width of such a groove(s) or ridge(s) may be between about 10 and about 500 microns, and in some embodiments, between about 100 and 300 microns, and in some embodiments, about 250 microns. Example of such embodiments are illustrated in FIGS. 3D and 3E.

In some embodiments, a portion of inner surface of the mold can be patterned, and in some embodiments, the entire inner surface (e.g., by laser micromachining), to create micro-features which, when replicated on the balloon's outer surface in the molding process, may achieve the desired functionality of the balloon. For example, in some embodiments, the surface roughness of the balloon may be modified for interaction with bodily tissues. In some embodiments, micro-cavities may be created along the balloon surface for the retention of drugs, and micro-grooves may be included for liquid drug flow enhancement.

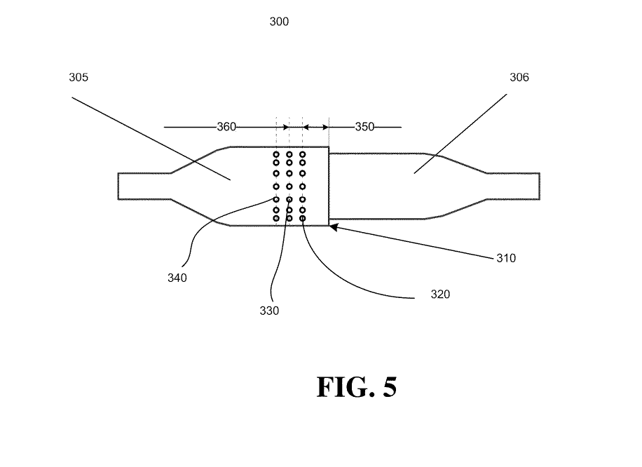

FIG. 5 illustrates an exemplary embodiment of the present disclosure, where balloon 200 includes a transition edge 310 between cylindrical portions 305, 306 (for example; see also, FIG. 3B), where laser machining has been effected. As shown, balloon 300 is placed approximately in the field of view of an operator or a vision system. Reference feature 310 is then located. Laser processing may then be performed at a location easily determined by a predetermined distance 350 away from the transition edge 310, to form, for example, a first laser pattern 320. Subsequent laser patterns 330 (e.g., at a distance 360 away from first reference pattern 320), and 340 may also be formed by referencing transition edge 310.

While some of the disclosed embody are presented for transversely split molds, for example, such concepts may equally be applicable for longitudinally split molds.

One of skill in the art will also appreciate that for embodiments where it has been disclosed that the inner surface of a mold is modified, it is also implied that such modifications can be made to one and/or another of the inner surface of the mold (e.g., metal mold) itself, and the inner surface of mold liner (if used). In any such embodiment, a feature or pattern on the inner surface of mold and/or liner is replicated on the outer surface of the balloon.

Any and all references to publications or other documents, including but not limited to, patents, patent applications, articles, webpages, books, etc., presented anywhere in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices, including any and all elements corresponding to medical device manufacturing. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference by specifically lacking one or more elements/features. In other words, claims to certain embodiments may contain one or more negative limitations to specifically exclude one or more elements/features resulting in embodiments which are patentably distinct from the prior art which include such features/elements.

What is claimed is:

1. A method for manufacturing a polymeric medical balloon comprising:
providing a mold, wherein the mold comprises a plurality of assembled parts configured for separation to remove a molded balloon;
configuring the interior surface of a least a portion of at least one part of the mold with one or more micro-features;
inserting a balloon preform within the mold;
expanding the preform within the mold to obtain an expanded balloon, wherein:
the one or more micro-features of the mold produce one or more corresponding features on the surface of the expanded balloon,
the one or more corresponding features on the surface of the expanded balloon effect at least one of a depth and height between about 10 μm and about 500 μm such that the one or more corresponding features are configured as reference points for positional registration of the balloon for subsequent processing of the expanded balloon;
and
removing the expanded balloon from the mold.

2. The method of claim 1, the one or more corresponding features on the surface of the expanded balloon effect at least one of a depth and height between about 50 μm and about 150 μm.

3. The method of claim 1, wherein the one or more corresponding features comprise one or more of a groove, a recess, and/or a protrusion.

4. The method of claim 3, wherein the one or more corresponding features comprise a plurality of radial and/or longitudinal grooves.

5. The method of claim 3, wherein the one or more micro-features which produce the one or more grooves, recesses, and/or protrusions on a balloon, are provided at or adjacent a seam between two parts of the mold.

6. The method of claim 3, wherein the one or more micro-features which produce the one or more grooves, recesses, and/or protrusions on a balloon, are provided at or adjacent a transitional area of the mold.

7. The method according to claim 3, wherein the grooves, recesses and/or protrusions comprise at least one of a cylindrical, spherical or otherwise round shape.

8. The method of claim 1, wherein subsequent processing includes at least one of:
modification of the balloon, and assembly of the balloon with other devices for a surgical procedure.

9. The method of claim 8, wherein the micro-features on the surface of the balloon are configured as one or more positional references for at least one of placing and attaching an expandable stent onto the balloon for surgical procedure.

10. The method of claim 8, wherein modification of the balloon comprises laser micromachining of the balloon.

11. The method of claim 1, wherein the one or more micro-features comprise a plurality of micro-features, and wherein some of the plurality of micro-features effect second corresponding features in the surface of the molded balloon which comprise drug depots.

12. A method for manufacturing a polymeric medical balloon comprising:
providing a mold, wherein the mold comprises a plurality of assembled parts configured for separation to remove a molded balloon, wherein:
a first part of the mold is arranged immediately adjacent a second part of the mold forming a seam therebetween in a balloon,
the first part of the mold is configured with a first radius at or adjacent the seam;
the second part of the mold is configured with a second radius at or adjacent the same;
the first radius is either larger or smaller than the second radius to effect a radial mis-match on a molded balloon;
inserting a balloon preform within the mold;
expanding the preform within the mold to obtain an expanded balloon;
wherein the radial mis-match establishes a measurable difference of between about 10 μm and about 500 μm in the balloon to establish a reference mark for positional registration of the balloon for subsequent processing of the expanded balloon; and
removing the expanded balloon from the mold.

13. The method of claim 12, wherein the difference between the first radius and the second radius is between about 50 μm and about 150 μm.

14. A polymeric balloon having one or more micro-features arranged on the surface of the balloon, the one or more micro-features effect at least one of a depth and height between about 10 μm and about 500 μm, wherein the one or more micro-features are configured as reference points for positional registration of the balloon for subsequent processing of the expanded balloon.

15. A polymeric balloon having at least two sections, a first section and a second section and a seam provided therebetween, wherein:
the first section of the balloon is configured with a first radius at or adjacent the seam,
the second section of the balloon is configured with a second radius at or adjacent the same,
the first radius is either larger or smaller than the second radius to effect a radial mis-match on the balloon, and
the radial mis-match establishes a measurable difference of between about 10 μm and about 500 μm to establish a reference mark for positional registration of the balloon for subsequent processing of the expanded balloon.
